(19) United States
(12) Patent Application Publication
(10) Pub. No.: US 2022/0370675 A1
(43) Pub. Date: Nov. 24, 2022

(54) Aroma Dispenser and Composition

(71) Applicant: Paulette Taylor, Peoria, AZ (US)

(72) Inventor: Paulette Taylor, Peoria, AZ (US)

(21) Appl. No.: 17/716,104

(22) Filed: Apr. 8, 2022

Related U.S. Application Data
(60) Provisional application No. 63/191,566, filed on May 21, 2021.

(51) Int. Cl. A61L 9/013; A61L 9/14

(52) U.S. Cl. CPC A61L 9/013; A61L 9/14; A61L 2209/21; A61L 2209/134

(57) ABSTRACT

The present invention relates to an herbal or sage extract composition for producing aroma without the need to burn the herb. The composition is in the form of an aerosol or a solid air freshener soaked in herbal solution. More specifically, the composition includes an herb such as sage extract that can be in the form of one of oil or powder, a propellant, an active ingredient and an organic solvent. The aerosol solution is sprayed on a surface and releases the herbal or sage aroma that helps in creating positive ambience and helps clearing negative energy. The composition can be used with an aerosol device and allows for a safer and more convenient way to produce the aroma.

### CROSS-REFERENCE TO RELATED APPLICATION

The present application claims priority to, and the benefit of, U.S. Provisional Application No. 63/191,566, which was filed on May 21, 2021, and is incorporated herein by reference in its entirety.

### FIELD OF THE INVENTION

The present invention relates generally to the field of air fresheners. More specifically, the present invention relates to an air freshening composition and product that applies the aroma or scent of sage and other herbs to any space without the need to burn real sage with an open flame or provide a smudge stick which, after lighting, produces smoke and the burned portions of the stick mark or otherwise damage items. The composition is suspended in a liquid solution which can be applied in any area via aerosol or pump spray or via a solid air freshener formed from a solidified composite from the liquid solution. The invention allows for a safer, efficient, and more convenient way to disperse and provide the sage aroma. Accordingly, the present disclosure makes specific reference thereto. Nonetheless, it is to be appreciated that aspects of the present invention are also equally applicable to other like applications, devices, and methods of manufacture.

### BACKGROUND

By way of background, sage is a staple herb in various cuisines around the world. Sage is also known for having a strong aroma and earthy flavor. It is a part of the mint family. Generally, sage has oval, dusty gray-green leaves with woody stems. Sage has been wrapped into bundles and secured by twine or other string forming a stick and can be ignited. Sage and other herbs have been used since ancient times for several purposes, such as warding off evil, dispersing negative energy, spiritual cleansing and other spiritual rituals. Smudge sticks are often ignited and the smoke from the smudge sticks fills the room and the ashes are used mark areas. Sage originated in the Mediterranean and was noted as being one of the most important herbs of that time period. Smudge sticks have been used by various indigenous populations and other cultures around the world.

Burning sage and other herbs have been fundamental to the practice of smudging to clear out spiritual impurities and pathogens and to create a positive energy. However, when sage is burned with an open flame, smoke is created along with aroma. Smoke typically contains dangerous particles and chemicals that can pose a health risk, especially when exposure occurs often. The smoke may cause health problems for individuals who are prone to asthma and other respiratory conditions. Often, people are required to open a window or door before and after burning the sage to eliminate the smoke. Sage, when burning, may be required to be removed from a room to prevent the space from being filled with too much smoke. Furthermore, a person may directly inhale the smoke which can create a problem of restlessness and breathlessness. People desire a source for providing a sage aroma that does not require the sage or other herbs, used in combination with the sage, to be burned for creating the aroma.

Often, people are required to dry sage and other herbs, such as lavender, cedar, sweetgrass, rosemary, rose, frankincense, myrrh, bayberry, as well as various combinations, which may be used along with sage before burning. People also need to trim sage, pull-out roots and other debris carefully so that stems are not cut. Such steps are not only time consuming, but the process also wastes sage which is economically detrimental for them. People desire a sage and herbal aroma source that is convenient and can be used anywhere.

Therefore, there exists a long-felt need in the art for a portable source of sage and herbal aroma that applies aroma of sage or other herbal combinations to any space without having to burn real sage or herbs with an open flame or other ignition device. There is also a long-felt need in the art for a sage or herbal aroma source that does not create smoke and the particles that commonly accompany the smoke. Additionally, there is a long-felt need in the art for a sage or herbal product that provides all the benefits of traditional sage or herb burning. Moreover, there is a long-felt need in the art for a sage, herbal, or combination product that eliminates having to collect, dry, trim or perform any other operations on real sage or herbs by a user. Further, there is a long-felt need in the art for a sage, herbal or combination aroma product that is easy to use and does not leave any residue after use. Finally, there is a long-felt need in the art for a sage, herbal or combination product that provides for a safer and more convenient way to produce the sage, herbal or combination aroma.

The subject matter disclosed and claimed herein, in one embodiment thereof, comprises a single-phase sage, herbal or combination extract aerosol composition for releasing sage, herbal or combination aroma without burning real sage or herbs. The single-phase aerosol composition can be applied to any surface via aerosol or pump spray and further includes a natural hydrocarbon propellant; a salvia blanca or divinorum extract active component; and at least an organic solvent. The active component concentration is from between 2 and 20 wt. %, preferably between 4 and 18 wt. % and more preferably between 6 and 15 wt. %. The organic solvent may be present in an amount between 20 and 60 percent with about 40 and 50 percent by weight being preferred. Other materials may be included to preserve freshness, such as salts, tocopherols, rosemary, ascorbic acid and combinations thereof. Other additives or materials for freshness may be present in an amount by weight of between 1 and 10 percent and more preferably between 3 and 7 percent. The composition may also include water, which may also be distilled water, in an amount of up to 85% by weight. Cyclodextrin may be included in an amount of 0.1 percent by weight to about 5% by weight. Additional herbs that may be used along with sage include, but are not limited to, lavender, cedar, sweetgrass, rosemary, rose, frankincense, myrrh, bayberry as well as various combinations of the foregoing.

In this manner, the sage aroma product of the present invention accomplishes all of the forgoing objectives and provides users with an extract of sage, herbal or combination suspended in a liquid solution for producing sage aroma without requiring burning sage or other herbs. The sage, herbal or combination solution is easy to be applied to any surface via aerosol, pump spray, or solid air freshener form containing the liquid composition. The solid may include gelatin or other stabilizers. The drops or mist of the sage, herbal or combination solution is dried to release sage and or other herbal aroma in a quick and effective manner.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a single-phase sage, herbal or combination extract aerosol composition. The single-phase aerosol composition further comprising a natural hydrocarbon propellant; a salvia blanca or divinorum extract active component; an organic solvent; the active component concentration is from between 2 and 20 wt. %, preferably between 4 and 18 wt. % and more preferably between 6 and 15 wt. %. The organic solvent may be present in an amount between 20 and 60 percent with about 40 and 50 percent by weight being preferred. Other materials may be included to preserve freshness, such as salts, tocopherols, rosemary, ascorbic acid and combinations thereof. Other additives or materials for freshness may be present in an amount by weight of between 1 and 10 percent and more preferably between 3 and 7 percent. The composition may also include water, which may also be distilled water, and may be present in an amount up to 85 percent by weight. Cyclodextrin may be included in an amount of 0.1 percent by weight to about 5% by weight. Additional herbs that may be used along with sage include, but are not limited to, lavender, cedar, sweetgrass, rosemary, rose, frankincense, myrrh, bayberry as well as various combinations of the foregoing.

In yet another embodiment, the propellant is selected from the group of natural hydrocarbons and natural fluorocarbons such as nitrogen in an amount by weight between 0.1 percent to 3 percent by weight.

In yet another embodiment, the organic solvent is isopropyl alcohol or ethanol, and the active component is extract of Salvia officinalis herb, salvia blanca or divinorum.

In yet another embodiment, an air-freshening sage aroma aerosol package is disclosed. The package includes an aerosol container storing a sage, herbal or other combination composition, a nozzle spray coupled to said container for dispensing said composition in the form of mist, the sage or herbal composition comprising from about 4 to 20 wt. % of sage oil; from about 10-25 wt. % of organic solvent; and from about 20-30 wt. % of propellant wherein the sage oil is extracted from steam distillation of leaves of Salvia officinalis herb.

In yet another embodiment, a combination of a solid air freshener soaked in a liquid medium for releasing sage aroma is disclosed. The solid air freshener includes a sage, herbal or other combination extract derived from steam distillation of leaves of an herb; and a solidifier selected from the group of paraffin, natural gum, and cetearyl alcohol. The liquid medium is sage or herbal oil aerosol, and the solid air freshener is soaked in the herbal oil aerosol and releases sage aroma to surroundings.

In yet another embodiment, a method for applying sage, herbal or other combination aroma to any space without having to burn real sage or herbs with an open flame is described. The method includes the steps of obtaining a sage or herbal aroma freshener composition comprising a fragrant product from about 4 to 20 wt. % of sage oil; an organic solvent from about 10-25 wt. %; and a propellant from about 20-30 wt. %; storing the composition in an aerosol container; directing the spray nozzle to a target area and spraying mist of composition in the form of droplets for releasing sage aroma.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a block diagram showing ingredients of one potential embodiment of the sage or herbal aroma composition of the present invention for releasing sage or other herbal aroma in accordance with the disclosed architecture;

FIG. 2 illustrates a block diagram showing ingredients of another potential embodiment of the sage or herbal aroma composition of the present invention for releasing sage or other herbal aroma in accordance with the disclosed architecture;

FIG. 3 illustrates a perspective view of an exemplary aerosol device of the present invention for releasing sage or other herbal aroma in accordance with the disclosed architecture; and

FIG. 4 illustrates a flow diagram showing exemplary steps performed in releasing sage or other herbal aroma to any space using the composition and sage aroma product of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a portable source of sage or herbal aroma that applies aroma of sage or other herbs to any space without having to burn real sage or other herbs with an open flame. There is also a long felt need in the art for a sage or other herbal aroma source that does not create smoke. Additionally, there is a long felt need in the art for a sage or other herbal product that provides all the benefits provided by traditional sage or herb burning. Moreover, there is a long felt need in the art for a sage or other herbal product that eliminates drying, trimming or performing any other operations to prepare smudge sticks or other herbal solutions by a user. Further, there is a long felt need in the art for a sage or other herbal aroma product that is easy to use and does not leave any residue after use. Finally, there is a long felt need in the art for a sage or herbal product that provides for a safer and more convenient way to produce the sage, herbal or combination aroma.

The present invention, in one exemplary embodiment, is a novel solid air freshener soaked in a liquid medium for releasing sage aroma. The solid air freshener includes a sage or herbal extract derived from steam distillation of leaves of an herb or combination of herbs; and a solidifier selected from the group of paraffin, natural gum, and cetearyl alcohol. The liquid medium is sage or other herbal oil aerosol, and the solid air freshener is soaked in the sage or herbal oil aerosol and releases sage or other herbal aroma to immediate surroundings.

In the present disclosure, an aerosol can be one of a large number of products which are dispensed as a mist, stream, spray, powder, foam or combinations thereof. Pressurized cans are the typical vehicle for the formation of aerosols to dispense an air freshener solution, providing a low cost, easy to use method of releasing the aroma. Typically, aerosol dispensers may include a container, which contains the extract or composition suspended in a liquid. A pressurized propellant is used to provide a force sufficient to discharge the liquid product from the container.

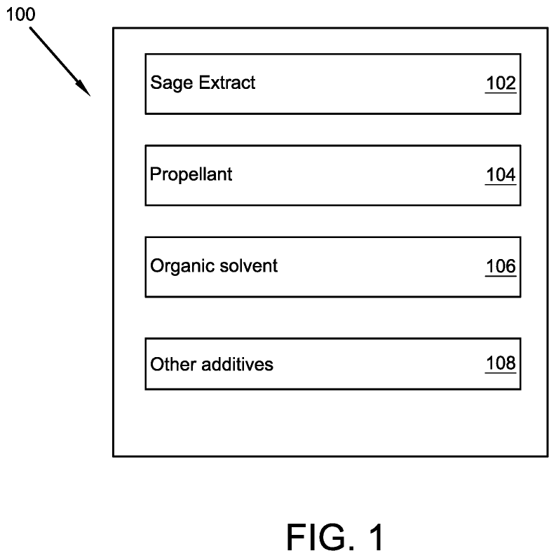

Referring initially to the drawings, FIG. 1 illustrates a block diagram showing ingredients of one potential embodiment of the sage aroma composition 100 of the present invention for releasing an herbal aroma in accordance with the disclosed specification. In the present embodiment, the composition 100 provides a user with the ability to disperse the herbal aroma to any space without having to burn the herbs with an open flame or other ignition source. More specifically, the composition is in the form of an aerosol that can be sprayed or dispersed via aerosol container or pump spray.

The composition 100 includes an herbal extract, such as a sage extract 102, that can be in the form of an herbal oil formed by steam distillation of leaves of the herb. Exemplary sage herbs may include white sage, garden sage, or ground sage. The herbal extract 102 alternatively can be in the form of powdered, ground, or rubbed leaves or other portions of the plants which are used to generate the aroma. A propellant 104, such as nitrogen, is used for driving the composition 100 out of an aerosol can or container. The composition 100 is preferably a single-phase aerosol composition that does not require a user to shake the aerosol container in which the composition 100 is stored before use. The propellant 104 is preferably a natural hydrocarbon but can be one or more of halogenated hydrocarbons, carbon dioxide, compressed air, compressed nitrogen, and combinations thereof.

An organic solvent 106 ingredient of the composition 100 is used for dissolving the herbal extract such as a sage extract 102 and the propellant 104. The organic solvent 106 can be one or more from a monohydric or polyhydric alcohol, acetone, ethanol, isopropyl alcohol or any other conventional solvent known in the art. It should be noted that the composition 100 can have any other natural essential oil or other additives 108, 210, such as water, for providing fragrance, preserving freshness or providing other desirable attributes.

In the composition 100 of the present embodiment, the herbal or sage extract 102 is from between 2 and 20 wt. %, preferably between 4 and 18 wt. % and more preferably between 6 and 15 wt. %. The propellant 104 is from about 20-30 wt. % and the organic solvent 106 is from about 10-25 wt. %. The organic solvent may be present in an amount between 20 and 60 percent with about 40 and 50 percent by weight being preferred. Other materials may be included to preserve freshness, such as salts, tocopherols, rosemary, ascorbic acid and combinations thereof. Other additives or materials for freshness may be present in an amount by weight of between 1 and 10 percent and more preferably between 3 and 7 percent. The composition may also include water, which may also be distilled water, up to 85% by weight. Cyclodextrin may be included in an amount of 0.1 percent by weight to about 5% by weight. Additional herbs that may be used along with sage include, but are not limited to, lavender, cedar, sweetgrass, rosemary, rose, frankincense, myrrh, bayberry as well as various combinations of the foregoing

The composition 100 releases sage or other herbal aroma that helps calming the mind and reduces anxiety. The composition 100 also cleanses spaces of negative energy and occurrences and attracts positive energy. The composition 100 is sprayed or otherwise dispensed in the form of mist or fine droplets that are dried easily to create an ambience of happiness.

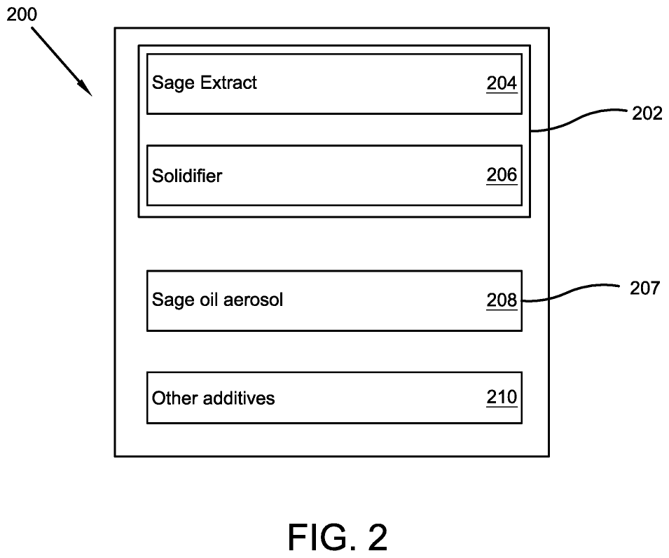

FIG. 2 illustrates a block diagram showing ingredients of another potential embodiment of the sage aroma composition of the present invention for releasing sage aroma in accordance with the disclosed architecture. In the present embodiment, the composition 200 is in the form of a solid air freshener 202 soaked in the herbal or sage oil aerosol 208 for releasing sage aroma in the environment. More specifically, the solid air freshener 202 includes herbal or sage extract 204 that can be in the form of powder or distilled form of herb leaves or plant. The herbal or sage extract 204 is combined with a solidifier 206 for forming the solid air freshener 202. The solidifier 206 can be one or more of paraffin, cetearyl alcohol, cellulose derivatives, and ether. The solidifier 206 helps in forming the solid air freshener 202 in the form of a solid gel bar that is soaked or alternatively dissolved in the herbal or sage oil aerosol 208. The herbal oil is provided in a reservoir or other container 207.

The composition 200 allows a user to place the soaked solid air freshener 202 at a place for releasing herbal or sage aroma in surroundings for creating a positive and fragrant ambience without requiring burning of sage. In the preferred embodiment, the solid air freshener 202 has solidifier 206 from between 25-45 wt. % and sage extract 204 from between 10-15 wt. %.

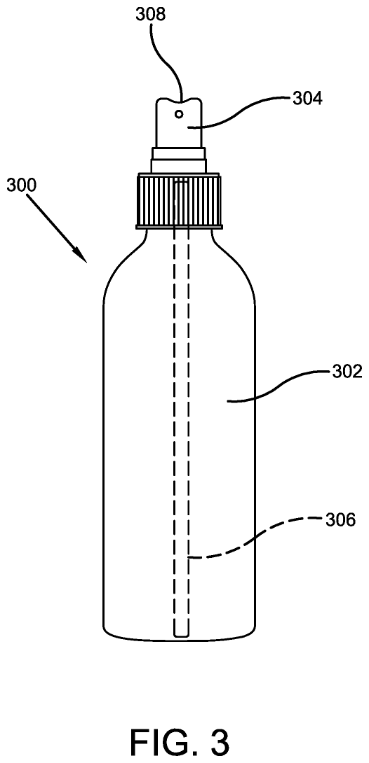

FIG. 3 illustrates a perspective view of an exemplary combination aerosol device having a reservoir of material of the present invention for releasing herbal or sage aroma in accordance with the disclosed specification. As illustrated, the aerosol spray device 300 includes an aerosol container or housing 302 for storing a reservoir of an herbal or sage aroma composition. A nozzle 304 is coupled to the aerosol container 302 through a suction pipe 306 such that when the trigger 308 is pulled or a button is depressed, the composition is released in the form of a mist and droplets to surroundings. The size of the mist droplets sprayed from the spray nozzle 304 of the aerosol device 300 is extremely fine and can range in the size from between 2 and about 15 microns, thus, the spray does not wet a surface and is easily dried.

The aerosol device 300 can be reusable or disposable. Composition solution of the present invention can also come in various packaging that can be used for refilling the aerosol device 300.

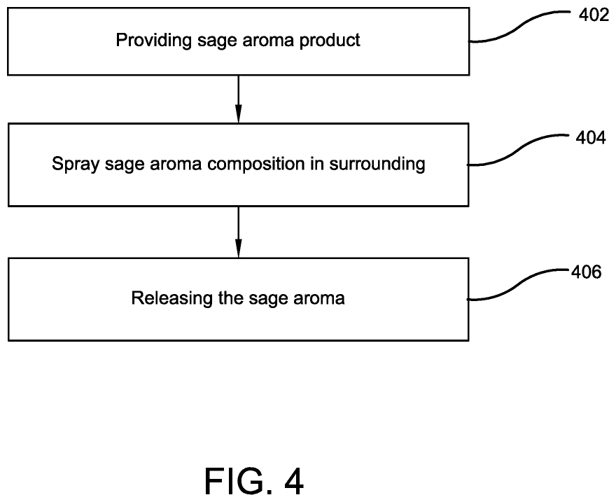

FIG. 4 illustrates a flow diagram showing exemplary steps performed in releasing sage or other herbal aroma to any space using the composition and sage or herbal aroma product of the present invention in accordance with the disclosed specification. As illustrated, initially, an herbal or sage aroma product having composition of the present invention is provided (Step 402). The sage or herbal aroma composition of the present invention is sprayed in the form of mist, droplets or like on a target surrounding area (Step 404). Finally, when the droplets get dried or evaporated, herbal or sage aroma is released and thus creating a positive ambience with clearing negative energy.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein, “sage or herbal aroma composition”, “composition”, and “air-freshening sage or herbal aroma composition” are interchangeable and refer to the air-freshening sage or herbal aroma composition 100, 200 of the present invention. Similarly, as used herein, “sage or herbal aroma aerosol device”, “aerosol device” and “sage or herbal aroma spray device” are interchangeable and refer to the sage aroma spray device 300 of the present invention.

Notwithstanding the forgoing, the air-freshening herbal or sage aroma composition 100, 200 and the aroma spray device 300 of the present invention can be of any suitable size and configuration as is known in the art without affecting the overall concept of the invention, provided that it accomplishes the above-stated objectives. One of ordinary skill in the art will appreciate that the air-freshening aroma composition 100, 200 and the aroma spray device 300 as shown in the FIGS. Are for illustrative purposes only, and that many other sizes and shapes of the air-freshening aroma composition 100, 200 and the aroma spray device 300 are well within the scope of the present disclosure. Although the dimensions of the air-freshening aroma composition 100, 200 and the aroma spray device 300 are important design parameters for user convenience, the air-freshening aroma composition 100, 200 and the aroma spray device 300 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An air freshening composition comprising:
an extract from an aroma producing herb provided in an amount ranging between 2 and 20 percent by weight;
an organic solvent in an amount ranging between 10 and 20 percent by weight; and
an active ingredient in an amount ranging between 2 and 20 percent by weight.

2. The air freshening composition as recited in claim 1, wherein the active ingredient is at least one of a salvia blanca, an officinalis or a divinorum.

3. The air freshening composition as recited in claim 1 further comprising a quantity of water or a quantity of distilled water in an amount up to 85 percent by weight.

4. The air freshening composition as recited in claim 1 further comprising a propellant ranging between 2 and 30 percent by weight.

5. The air freshening composition as recited in claim 1, wherein the aroma producing herb is a sage.

6. The air freshening composition as recited in claim 1 further comprising at least one additional additive.

7. The air freshening composition as recited in claim 6, wherein the at least one additional additive is an herb selected from the group consisting of a lavender, a cedar, a sweetgrass, a rosemary, a rose, a frankincense, a myrrh, a bayberry and a combination thereof.

8. The air freshening composition as recited in claim 6, wherein the at least one additional additive is a cyclodextrin in an amount ranging between 0.1 and 5 percent by weight.

9. The air freshening composition as recited in claim 6, wherein the at least one additional additive is a freshness preservative.

10. The air freshening composition as recited in claim 9, wherein freshness preservative is selected from a group consisting of a salt, a tocopherol, a rosemary, an ascorbic acid and a combination thereof.

11. The air freshening composition as recited in claim 4, wherein the propellant is at least one of a carbon dioxide, a compressed air, a compressed nitrogen or a combination thereof.

12. The air freshening composition as recited in claim 1, wherein the organic solvent is an isopropyl alcohol or an ethanol.

13. An air freshener solution and dispenser combination comprising:
an air freshener solution comprised of an active ingredient, an herb and an organic solvent;
a propellant; and
a container for dispensing the air freshener solution and comprising a reservoir holding the air freshener solution, wherein the active ingredient is comprised of at least one of a salvia blanca, an officinalis or a divinorum.

14. The air freshener solution and dispenser combination as recited in claim 13, wherein the organic solvent is an isopropyl alcohol or an ethanol.

15. The air freshener solution and dispenser combination as recited in claim 13, wherein the propellant is comprised of at least one of a carbon dioxide, a compressed air or a compressed nitrogen.

16. The air freshener solution and dispenser combination as recited in claim 13, wherein the air freshener solution further comprises at least one additive.

17. The air freshener solution and dispenser combination as recited in claim 13, wherein the herb is selected from a group consisting of a sage, a lavender, a cedar, a sweetgrass, a rosemary, a rose, a frankincense, a myrrh, a bayberry and a combination thereof.

18. The air freshener solution and dispenser combination as recited in claim 16, wherein the at least one additive is a freshness preservative.

19. The air freshener solution and dispenser combination as recited in claim 17, wherein the freshness preservative is selected from a group consisting of a salt, a tocopherol, a rosemary, an ascorbic acid and a combination thereof.

20. A single phase aerosol solution comprising:
a sage oil extract in an amount ranging between 2 and 20 percent by weight;
an isopropyl alcohol;
a propellant selected from a group consisting of a carbon dioxide, a compressed air, a compressed nitrogen and a combination thereof; and
an active ingredient selected from a group consisting of a salvia blanca, an officinalis or a divinorum.
